(19) United States
(12) Patent Application Publication
(10) Pub. No.: US 2025/0025523 A1
(43) Pub. Date: Jan. 23, 2025

(54) COMPOSITION FOR PREVENTING OR TREATING MUSCLE DISEASE COMPRISING SARGASSUM SERRATIFOLIUM EXTRACT

(71) Applicant: PUKYONG NATIONAL UNIVERSITY INDUSTRY- UNIVERSITY COOPERATION FOUNDATION, Busan (KR)

(72) Inventors: Bonggi LEE, Busan (KR); Hyeung-Rak KIM, Busan (KR); Min-Kyeong LEE, Busan (KR); Myeong-Jin KIM, Busan (KR); Jiwon BAEK, Busan (KR); Heeyeon RYU, Ulsan (KR)

(73) Assignee: PUKYONG NATIONAL UNIVERSITY INDUSTRY- UNIVERSITY COOPERATION FOUNDATION, Busan (KR)

(21) Appl. No.: 18/713,025

(22) PCT Filed: Nov. 23, 2022
(86) PCT No.: PCT/KR2022/018616

§ 371 Date: May 23, 2024

(30) Foreign Application Priority Data
Nov. 23, 2021 (KR) 10-2021-0161925

(51) Int. Cl. A61K 36/03; A61P 21/00

(52) U.S. Cl. CPC A61K 36/03; A61P 21/00

(57) ABSTRACT

The present invention relates to a composition for the prevention or treatment of muscle diseases comprising a Sargassum serratifolium extract. The Sargassum serratifolium extract of the present invention has been found to protect muscle cells from dexamethasone-induced atrophy and muscle loss caused by the side effects of atorvastatin, inhibit the reduction in myotube diameter, suppress muscle atrophy, induce muscle hypertrophy, and inhibit muscle protein degradation. Additionally, in an animal model with dexamethasone-induced muscle atrophy, it was confirmed to increase muscle strength, improve muscle function, and increase muscle mass, making it useful for the prevention or treatment of muscle diseases.

### TECHNICAL FIELD

The present invention relates to a composition for preventing or treating muscle diseases comprising Sargassum serratifolium extracts.

### BACKGROUND ART

The skeletal muscle is the largest metabolic organ, which accounts for about 40% to 50% of the body weight, and is a protein reservoir, which controls physical movement, and also maintains various physical functions such as energy consumption, respiration, glucose, lipids, and amino acid homeostasis control. The skeletal muscle amount is a factor that determines muscle strength, muscle endurance strength, and physical performance ability, and when the muscle skeletal mass, size, and strength are excessively lost, skeletal muscle weakness and atrophy are induced. Among them, the skeletal muscle is the largest organization in our body, which accounts for about 40% to 50% of the body weight of normal individuals, and is responsible for essential functions such as exercise, respiration, and heartbeats (Int J Biochem Cell Biol 37:1985-1996, 2005; Pharmacol Rev 61:373-393, 2009; Calcif Tissue Int 96:183-195, 2015).

Likewise, in humans, the amount of muscle, which is crucial for bodily functions, starts to decrease by 1-2% per year after the age of 50. By the time they reach 80, the muscle mass significantly decreases to about half of the maximum amount. Consequently, many elderly individuals experience sarcopenia or muscle atrophy, leading to a decline in physical function, disabilities, and frequent falls, all of which result in a reduced quality of life (Korean J Food and Nutr 4:133-139, 1991; J Nutr 127: 990S-991S, 1997; Curr Aging Sci 1:182-191, 2008). In particular, as the average lifespan increases and the proportion of the elderly people increases globally, muscle atrophy is recognized as an important aging disease (Curr Opin Clin Nutr Metab Care 13:1-7, 2010). Normally, muscle mass in the human body is maintained at a constant level through a balance of protein synthesis and degradation. However, when protein degradation increases or synthesis decreases, muscle atrophy occurs. Muscle atrophy is commonly observed not only with aging, malnutrition, and reduced physical activity but also in chronic wasting diseases such as cancer, diabetes, uremia, chronic pulmonary disease, heart failure, and AIDS. It also occurs in acute conditions such as sepsis, surgery, trauma, and burns (Int J Biochem Cell Biol 37:2156-68, 2005). The muscular atrophy is caused by a gradual decrease in muscle amount, and refers to muscle weakness and degeneration, and is caused by various pathological conditions such as activity decline, nutritional deficiency, denervation, burns, chronic diseases, steroids, and aging, and is accompanied by muscle weakness in most cases. The muscular atrophy is promoted by inactivity, oxidative stress, and chronic inflammation, weakens muscle function and exercise ability, causing significant disability in normal daily life ability, and limits independent life by increasing the risk of falls and fracture. The most important factor that determines muscle function is muscle mass, which is maintained by the balance of protein synthesis and degradation. The muscle atrophy occurs when protein degradation occurs more than synthesis. The muscle size is regulated by the intracellular signaling pathways that induce anabolic or catabolic activity that occurs in the muscle, and the muscle protein synthesis is increased when more signaling reactions that induce synthesis rather than the degradation of muscle proteins. In addition, it has been reported that the occurrence of muscle atrophy is related to the increase in the expression of proteins such as Atrogin-1 and Murf-1 that promote the degradation of myofibrillar proteins, and the inhibition of the PI3K-Akt-mTOR signaling pathway involved in muscle-related protein synthesis (Am J Physiol Cell Physiol 287: C834-843, 2004). Currently, there are no clear therapeutic drugs available on the market to treat or improve muscle atrophy caused by chronic diseases and aging, and we rely solely on dietary supplements like protein supplements. The most fundamental and effective treatments for muscle atrophy are resistance exercises and aerobic exercises. However, regular exercise is highly challenging for severely ill patients, the elderly, and individuals with disabilities, making acceptance very low. Therefore, it is urgent to develop compositions derived from natural substances for the prevention and improvement of muscle atrophy that do not pose issues of tolerance or safety (Proc Natl Acad Sci USA 98:14440-14445, 2001; J Biol Chem 280:2737-2744, 2005).

Meanwhile, Sargassum serratifolium (C. Agardh) C. Agardh 1820 is a perennial brown algae predominantly found along the southern and southeastern coasts of Korea and the coastal areas of Jeju Island. It is a large brown algae, with plant bodies ranging from 1 to 4 meters in length, and has roots that are conical and 4-5 cm in diameter with growth rings. The stem is cylindrical and short, branching into numerous central branches, with double serrated edges on the stem leaves as a morphological characteristic. It is known to possess various biological activities such as liver protection, improvement of serum lipids, anti-obesity effects, wrinkle improvement, and skin whitening. However, research on its activity for the prevention and treatment of muscle atrophy has not been reported so far.

### DETAILED DESCRIPTION

### Technical Problem

An object of the present invention is to provide a pharmaceutical composition for preventing or treating muscle disease comprising Sargassum serratifolium extract as an active ingredient.

In addition, another object of the present invention is to provide a food composition for preventing or improving muscle disease comprising Sargassum serratifolium extract as an active ingredient.

In addition, yet another object of the present invention is to provide a pharmaceutical composition for promoting muscle differentiation, muscle regeneration, or strengthening muscle comprising Sargassum serratifolium extract as an active ingredient.

In addition, yet another object of the present invention is to provide a food composition for promoting muscle differentiation, muscle regeneration, or muscle strengthening comprising Sargassum serratifolium extract as an active ingredient.

In addition, yet another object of the present invention is to provide a pharmaceutical composition for increasing muscle mass or promoting muscle growth comprising Sargassum serratifolium extract as an active ingredient.

In addition, yet another object of the present invention is to provide a food composition for increasing muscle mass or promoting muscle growth comprising Sargassum serratifolium extract as an active ingredient.

In addition, yet another object of the present invention is to provide a food composition for improving muscle function comprising Sargassum serratifolium extract as an active ingredient.

In addition, yet another object of the present invention is to provide a cosmetic composition for improving muscle function comprising Sargassum serratifolium extract as an active ingredient.

In addition, yet another object of the present invention is to provide a method of treating muscle disease comprising administering a pharmaceutically effective amount of Sargassum serratifolium extract to a subject.

### Technical Solution

In order to achieve the object, the present invention provides a pharmaceutical composition for preventing or treating muscle disease comprising Sargassum serratifolium extract as an active ingredient.

In addition, another aspect of the present invention provides a food composition for preventing or improving muscle disease comprising Sargassum serratifolium extract as an active ingredient.

In addition, yet another aspect of the present invention provides a pharmaceutical composition for promoting muscle differentiation, muscle regeneration, or strengthening muscle comprising Sargassum serratifolium extract as an active ingredient.

In addition, yet another aspect of the present invention provides a food composition for promoting muscle differentiation, muscle regeneration, or strengthening muscle comprising Sargassum serratifolium extract as an active ingredient.

In addition, yet another aspect of the present invention provides a pharmaceutical composition for increasing muscle mass or promoting muscle growth comprising Sargassum serratifolium extract as an active ingredient.

In addition, yet another aspect of the present invention provides a food composition for increasing muscle mass or promoting muscle growth comprising Sargassum serratifolium extract as an active ingredient.

In addition, yet another aspect of the present invention provides a food composition for improving muscle function comprising Sargassum serratifolium extract as an active ingredient.

In addition, yet another aspect of the present invention provides a cosmetic composition for improving muscle function comprising Sargassum serratifolium extract as an active ingredient.

In addition, yet another aspect of the present invention provides a method of treating muscle disease comprising administering a pharmaceutically effective amount of Sargassum serratifolium extract to an individual.

### Advantageous Effects

According to the present invention, the extract of Sargassum serratifolium protects muscle cells, inhibits the reduction of myotube diameter, suppresses muscle atrophy, induces muscle hypertrophy, and inhibits the degradation of muscle proteins from muscle atrophy induced by dexamethasone and the side effects of atorvastatin. Additionally, in an animal model where muscle atrophy was induced by dexamethasone, it was confirmed to increase muscle strength, improve muscle function, and increase muscle mass. Therefore, it can be effectively utilized for the prevention or treatment of muscle diseases.

### DESCRIPTION OF DRAWINGS

FIG. 1 is a diagram showing the cytotoxicity of the Sargassum serratifolium ethanol extract on C2C12 myotubes.

FIG. 2 is a diagram showing the changes in cell viability due to the treatment with the Sargassum serratifolium ethanol extract in C2C12 myotubes treated with dexamethasone.

FIG. 3A is a diagram showing the inhibitory effect of the Sargassum serratifolium ethanol extract on the reduction of myotube diameter in C2C12 myotubes treated with dexamethasone to induce muscle atrophy, confirmed by Giemsa-Wright staining.

FIG. 3B is a diagram quantifying the myotube diameter in C2C12 myotubes treated with dexamethasone to induce muscle atrophy and the effect of the Sargassum serratifolium ethanol extract.

FIG. 4 is a diagram showing the level of Akt phosphorylation in C2C12 myotubes treated with dexamethasone to induce muscle atrophy, confirmed by Western blot analysis.

FIG. 5A is a diagram quantifying the mRNA expression of atrogin-1/MAFbx in C2C12 myotubes induced with dexamethasone to cause muscle atrophy, treated with the Sargassum serratifolium ethanol extract.

FIG. 5B is a diagram quantifying the mRNA expression of MuRF1 in C2C12 myotubes induced with dexamethasone to cause muscle atrophy, treated with the Sargassum serratifolium ethanol extract.

FIG. 5C is a diagram confirming the changes in protein expression levels of atrogin-1/MAFbx and MuRF1 induced by dexamethasone treatment, treated with the Sargassum serratifolium ethanol extract, using Western blot analysis.

FIG. 6A is a diagram quantifying the mRNA expression of atrogin-1/MAFbx in C2C12 myotubes induced with atorvastatin to cause muscle atrophy, treated with the Sargassum serratifolium ethanol extract.

FIG. 6B is a diagram quantifying the mRNA expression of MuRF1 in C2C12 myotubes induced with atorvastatin to cause muscle atrophy, treated with the Sargassum serratifolium ethanol extract.

FIG. 7A is a diagram showing the effect of the Sargassum serratifolium ethanol extract on increasing muscle strength in mice with muscle atrophy, measured by Grip strength.

FIG. 7B is a diagram showing the effect of the Sargassum serratifolium ethanol extract on increasing muscle strength in mice with muscle atrophy, measured by Maximum weight.

FIG. 8A is a diagram showing the effect of the Sargassum serratifolium ethanol extract on improving muscle function in mice with muscle atrophy, measured by maximum speed in a treadmill test.

FIG. 8B is a diagram showing the effect of the Sargassum serratifolium ethanol extract on improving muscle function in mice with muscle atrophy, measured by running time in a treadmill test.

FIG. 8C is a diagram showing the effect of the Sargassum serratifolium ethanol extract on improving muscle function in mice with muscle atrophy, measured by running distance in a treadmill test.

FIG. 9A is a diagram showing the increase in lean body mass using Lean body mass/Total mass in mice with muscle atrophy, treated with the Sargassum serratifolium ethanol extract.

FIG. 9B is a diagram showing the decrease in fat mass using Fat mass/Total mass in mice with muscle atrophy, treated with the Sargassum serratifolium ethanol extract.

FIG. 10 is a diagram showing the effect of the Sargassum serratifolium ethanol extract on increasing muscle mass measured by calf thickness in mice with muscle atrophy.

### BEST MODE OF THE INVENTION

Hereinafter, embodiments of the present invention will be described in detail with reference to the accompanying drawings. However, the following embodiments are presented as examples of the present invention, and detailed descriptions of well-known techniques or constructions may be omitted when it is judged that they may unnecessarily obscure the gist of the present invention, and the present invention is not limited thereto. The present invention can be modified and applied in various ways within the scope of the appended claims and the equivalents interpreted therefrom.

Further, terminologies used herein are terminologies used to properly express preferred examples of the present invention, which may vary according to a user, an operator's intention, or customs in the art to which the present invention pertains. Therefore, these terminologies used herein will be defined based on the contents throughout the specification. Throughout the specification, unless explicitly described to the contrary, when a certain part “comprises” a certain component, it will be understood to imply the inclusion of stated elements but not the exclusion of any other elements.

Throughout this specification, “%” used to indicate the concentration of a specific substance is solid/solid in % (w/w), solid/liquid in % (w/v), and liquid/liquid in % (v/v), unless otherwise specified.

In one aspect, the present invention relates to a pharmaceutical composition for preventing or treating muscle disease comprising Sargassum serratifolium extract as an active ingredient.

In one embodiment, the extract of Sargassum serratifolium may be extracted with one or more solvents selected from a group consisting of water, organic solvents with carbon numbers ranging from 1 to 6, supercritical fluids, and subcritical fluids. The organic solvents with carbon numbers ranging from 1 to 6 may include, but are not limited to, alcohols, acetone, ethers, benzene, chloroform, ethyl acetate, methylene chloride, hexane, cyclohexane, and petroleum ether, and alcohols with carbon numbers ranging from 1 to 6 are particularly preferable.

In one embodiment, the muscle disease may be one or more selected from the group consisting of atony, muscular atrophy, muscular dystrophy, muscle degeneration, myasthenia, cachexia, and sarcopenia, and is preferably muscular atrophy, and is most preferably muscular atrophy caused by dexamethasone or atorvastatin.

In one embodiment, the Sargassum serratifolium extract may protect muscle cells, and particularly may protect muscle cells against dexamethasone.

In one embodiment, the Sargassum serratifolium extract may inhibits myotube diameter reduction, and the myotube diameter reduction may be caused by muscular atrophy caused by dexamethasone.

In one embodiment, the Sargassum serratifolium extract may increase phosphorylation of Akt, thereby inhibiting muscle atrophy and causing muscle hypertrophy.

In one embodiment, the Sargassum serratifolium extract can inhibit mRNA and protein expression of atrogin-1/MAFbx or MuRF1, thereby inhibiting the degradation of muscle proteins.

In one embodiment, the Sargassum serratifolium extract can increase muscle strength, and the increase of muscle strength can increase the grip strength.

In one embodiment, the Sargassum serratifolium extract can improve muscle function, and the muscle function can be muscular endurance.

In one embodiment, the Sargassum serratifolium extract can increase muscle mass relative to body weight.

The term “extract” used herein refers to an active ingredient separated from a natural product, that is, a substance that exhibits the desired activity. The extract can be obtained by a extraction process using water, an organic solvent, or a mixed solvent thereof, and includes the dry powder of the extract or any form formulated using the same. In addition, the extract includes fractionating the extract that has undergone the extraction process. The method of extracting the extract is not particularly limited, and can be extracted by a method such as stirring, shaking, hot water, cold needle, reflux cooling, or ultrasonic extraction. As the extraction solvent, water, a polar solvent such as a lower alcohol of c1-c4, or a non-polar solvent such as hexane, chloroform, dichloromethane, or ethyl acetate, or a mixture of two or more thereof can be used.

The composition of the present invention can be prepared as a pharmaceutical composition for preventing or treating muscle disease by additionally containing other active ingredients having the same or similar function as the Sargassum serratifolium extract, or additionally containing other active ingredients having a different function from the above ingredients.

In the present invention, the term “prevention” refers to any action that inhibits or delays the occurrence, spread, and recurrence of muscle disease by administration of the pharmaceutical composition according to the present invention, and the term “treatment” refers to any action that improves or advantageously changes the symptoms of muscle disease by administration of the composition of the present invention. A person having ordinary skill in the art to which the present invention pertains the exact criteria of the diseases effective in the composition of the present invention by referring to the data presented by the Korean Medical Association, and can determine the improvement, improvement, and degree of treatment.

The term “therapeutically effective amount” used in combination with the active ingredients in the present invention refers to an amount effective in preventing or treating muscle disease, and the therapeutically effective amount of the composition of the present invention may vary depending on various factors, for example, the administration method, the target site, and the condition of the patient. Therefore, when used in humans, the dosage should be determined as appropriate in consideration of safety and efficacy. It is also possible to estimate the amount used in humans from the effective amount determined through animal experiment. These things are described, for example, Hardman and Limbird, eds., Goodman and Gilman's The Pharmacological Basis of Therapeutics, 10th ed. (2001), Pergamon Press; and E. W. Martin ed., ed. Remington's Pharmaceutical Sciences, 18th ed. (1990), Mack Publishing Co.

The pharmaceutical composition of the present invention is administered in a pharmaceutically effective amount. As used herein, the term “pharmaceutically effective amount” refers to an amount that is sufficient to treat a disease and does not cause side effects at a reasonable benefits/risk ratio applicable to medical treatment, and the effective dose level may be determined according to the factors including the patient's health status, the cause of the disease of the muscle disease, the severity, the activity of the drug, the sensitivity to the drug, the method of administration, the time of administration, the route of administration and the rate of excretion, the treatment period, the drugs used in combination or simultaneously, and other factors well known in the medical field. The composition of the present invention may be administered as an individual therapeutic agent or in combination with other therapeutic agents, may be administered sequentially or simultaneously with conventional therapeutic agents, and may be administered either a single or multiple doses. In consideration of all of the above factors, it is important to administer an amount capable of obtaining a maximum effect with a minimum amount without side effects, and this may be easily determined by those skilled in the art.

The pharmaceutical composition of the present invention may include a carrier, a diluent, an excipient, or a combination of two or more thereof conventionally used in biological preparations. As used herein, the term “pharmaceutically acceptable” refers to a property that is not toxic to the cells or humans exposed to the composition. The carrier is not particularly limited as long as it is suitable for the composition to be delivered in vivo, and for example, the compound described in Merck Index, 13th ed., Merck & Co., Inc., saline, sterile water, Ringer's solution, buffered saline, dextrose solution, maltodextrin solution, glycerol, ethanol, and one or more of these components may be mixed and used, and other conventional additives such as antioxidants, buffers, and bacteriostatic agents may be added as necessary. In addition, the diluent, dispersant, surfactant, binder, and lubricants may be additionally added to be formulated into main use formulations such as aqueous solutions, suspensions, emulsions, and the like, pills, capsules, granules, or tablets. Furthermore, the pharmaceutical composition may be preferably formulated according to each disease or ingredients by appropriate methods in the art or using the method disclosed in Remington's Pharmaceutical Science (Mack Publishing Company, Easton PA, 18th, 1990).

In one embodiment, the pharmaceutical composition may be selected from a group consisting of oral formulations, topical formulations, suppositories, sterile injectable solutions, and sprays, with oral or injectable formulations being particularly preferable.

As used herein, the term “administration” refers to delivering a specified substance to an individual or patient by any suitable means. Depending on the intended method, administration can be either non-oral (such as intravenous, subcutaneous, intraperitoneal, or local injection formulations) or oral. The dosage may vary depending on factors such as the patient's weight, age, sex, health condition, diet, time of administration, method of administration, elimination rate, and severity of the condition. Liquid formulations for oral administration of compositions of the present invention include suspensions, solutions, emulsions, syrups, etc. In addition to commonly used simple diluents such as water, liquid paraffin, various excipients such as wetting agents, sweeteners, flavoring agents, preservatives, etc., may also be included. Formulations for non-oral administration may include sterilized aqueous solutions, non-aqueous solvents, suspensions, emulsions, freeze-dried formulations, suppositories, etc. The pharmaceutical composition of the present invention may also be administered by any device capable of delivering the active substance to target cells. Preferred administration routes and formulations include intravenous injections, subcutaneous injections, intraperitoneal injections, intramuscular injections, and topical injections. Injectable formulations may be prepared using aqueous solvents such as saline, Ringer's solution, plant oils, high-grade fatty acid esters (e.g., ethyl oleate), alcohols (e.g., ethanol, benzyl alcohol, propylene glycol, glycerin), and non-aqueous solvents, along with stabilizers (e.g., ascorbic acid, sodium hydrogen sulfite, sodium thiosulfate, BHA, tocopherol, EDTA), emulsifiers, buffering agents for pH adjustment, and pharmaceutical carriers to inhibit microbial growth (e.g., silver nitrate, chlorhexidine, benzalkonium chloride, phenol, cresol, benzyl alcohol).

As used herein, the term “subject” refers to all animals including monkeys, cows, horses, sheep, pigs, chickens, turkeys, quails, cats, dogs, mice, rats, rabbits, or guinea pigs, which include humans who have or can develop the muscle disease, and by administering the pharmaceutical composition of the present invention to the subject, the diseases may be effectively prevented or treated. The pharmaceutical composition of the present invention may be administered in parallel with the existing therapeutic agent.

The pharmaceutical composition of the present invention may further include pharmaceutically acceptable additives, wherein pharmaceutically acceptable additives may include starch, pregelatinized starch, microcrystalline cellulose, lactose, povidone, colloidal silicon dioxide, calcium hydrogen phosphate, lactose, mannitol, molasses, arabic gum, pregelatinized starch, corn starch, powdered cellulose, hydroxypropyl cellulose, Opadry, sodium starch glycolate, carnauba wax, synthetic aluminum silicate, stearic acid, magnesium stearate, aluminum stearate, calcium stearate, dextrose, sorbitol, and talc. Pharmaceutically acceptable additives according to the present invention may preferably be included in the composition in an amount ranging from 0.1 to 90 parts by weight, although not limited thereto.

In one aspect, the present invention relates to a food composition for preventing or improving muscle disease comprising the Sargassum serratifolium extract as an active ingredient.

In one embodiment, the Sargassum serratifolium extract may be extracted using one or more solvents selected from a group consisting of water, organic solvents containing 1 to 6 carbon atoms, amine-based fluids, and supercritical fluids. The organic solvent having a carbon number of 1 to 6 may be one or more selected from the group consisting of C1-6 alcohols, acetone, ethers, benzene, chloroform, ethyl acetate, methylene chloride, hexane, cyclohexane, and petroleum ether, and C1-6 alcohols are more preferred.

In one embodiment, muscle disorders may include a group consisting of atony, muscular atrophy, muscular dystrophy, muscle degeneration, myasthenia, cachexia, and sarcopenia, with muscular atrophy being preferable, and muscular atrophy induced by dexamethasone or atorvastatin being most preferable.

In case of using the composition of the present invention as a food composition, the Sargassum serratifolium extract can either be directly added or utilized in conjunction with other food or food ingredients, and it can be applied appropriately following conventional methods. The formulation may incorporate food adjuncts that are food-grade and permissible, with the quantity of active ingredient being determined accordingly depending on the intended use (preventive, health, or therapeutic intervention).

The term “food supplement additive” used in the present invention means a component that can be supplemented to food, and is added to prepare health functional foods of each formulation, and can be appropriately selected by those skilled in the art. Examples of food supplement additives include various nutrients, vitamins, minerals (electrolytes), flavoring agents such as synthetic and natural flavors, coloring and fillers, pectic acid and its salts, alginic acid and its salts, organic acids, protective colloidal thickeners, pH adjusting agents, stabilizers, preservatives, glycerin, alcohols, and carbonated agents used in carbonated beverages, but the types of food supplement additives of the present invention are not limited by the above examples.

The food composition of the present invention may include a health functional food. The term “health functional food” used in the present invention refers to foods prepared and processed in the form of tablets, capsules, powders, granules, liquids, and pills using raw materials or ingredients having useful functionality for the human body. Here, “functional” means adjusting nutrients for the structure and function of the human body or obtaining a useful effect for health use such as physiological action. The health functional food of the present invention may be manufactured by a method commonly used in the ordinary technical field, and in the manufacturing, a raw material and components commonly added in the ordinary technical field may be added to prepare it. In addition, the formulation of the health functional food may also be manufactured without limitation as long as it is recognized as a health functional food. The food composition of the present invention can be prepared in various forms and has advantages in that it does not have side effects that may occur when taking a long-term medicine using seaweed as a raw material, unlike general medicine, and has excellent portability, and the health functional food of the present invention can be ingested as an adjuvant to enhance the effect of a muscle disease therapeutic agent.

In addition, there is no limitation on the type of health food that can be used with the composition of the present invention. In addition, the composition containing the Sargassum serratifolium extract of the present invention as an active ingredient can be prepared by mixing appropriate other auxiliary ingredients that can be contained in health functional foods and known additives according to the selection of those skilled in the art. Examples of foods that can be added include meat, sausage, bread, chocolate, candies, snacks, confectionery, pizza, ramen, other noodles, gums, dairy products including ice cream, various soups, beverages, tea, drinks, alcoholic beverages, and vitamin complexes, and can be prepared by adding the extract according to the present invention as a main component to juice, tea, jelly and juice.

In addition, the present invention provides a pharmaceutical composition for promoting muscle differentiation, muscle regeneration or muscle strengthening comprising the Sargassum serratifolium extract as an active ingredient.

In addition, the present invention provides a food composition for promoting muscle differentiation, muscle regeneration or muscle strengthening comprising Sargassum serratifolium extract as an active ingredient.

In addition, the present invention provides a pharmaceutical composition for increasing muscle mass or promoting muscle growth comprising Sargassum serratifolium extract as an active ingredient.

In addition, the present invention provides a food composition for increasing muscle mass or promoting muscle growth comprising the Sargassum serratifolium extract as an active ingredient.

In addition, the present invention provides a food composition for improving muscle function comprising the Sargassum serratifolium extract as an active ingredient.

In addition, the present invention provides a cosmetic composition for improving muscle function comprising the Sargassum serratifolium extract as an active ingredient.

The cosmetic compositions containing the active ingredients of the present invention can be utilized for various purposes related to muscle function enhancement. Products to which this composition can be added include, for example, various cosmetics such as creams, lotions, toners, essences, as well as hair care products like shampoos, conditioners, cleansers, facial cleansers, soaps, treatments, masks, and beauty essences.

The cosmetic formulations of the present invention comprise compositions selected from a group consisting of water-soluble vitamins, fat-soluble vitamins, polymer peptides, polymer saccharides, sphingolipids, and seaweed extracts.

As for water-soluble vitamins, any vitamin that can be blended into cosmetics is acceptable, but preferably, vitamins B1, B2, B6, pyridoxine, pyridoxine hydrochloride, vitamin B12, pantothenic acid, niacin, niacinamide, folic acid, vitamin C, vitamin H, and their salts (such as thiamine hydrochloride, sodium ascorbate, etc.) or derivatives (such as sodium ascorbyl phosphate, magnesium ascorbyl phosphate, etc.) can be included in the water-soluble vitamins used in the present invention. Water-soluble vitamins can be obtained by conventional methods such as microbial conversion, purification from microbial cultures, enzymatic methods, or chemical synthesis.

As for fat-soluble vitamins, any vitamin that can be blended into cosmetics is acceptable, but preferably, vitamins A, carotene, vitamin D2, vitamin D3, vitamin E (d1-alpha tocopherol, d-alpha tocopherol, d-alpha tocopherol), and their derivatives (such as palmitate ascorbic, stearate ascorbic, dipalmitate ascorbic, acetate dl-alpha tocopherol, niacin dl-alpha tocopherol vitamin E, DL-panthenol alcohol, D-panthenol alcohol, panthenol ethyl ether, etc.) can be included in the fat-soluble vitamins used in the present invention. Fat-soluble vitamins can be obtained by conventional methods such as microbial conversion, purification from microbial cultures, enzymatic methods, or chemical synthesis.

The polymer peptide may be any one that can be blended in cosmetics, but preferably include collagen, hydrolytic collagen, gelatin, elastin, hydrolytic elastin, keratin, and the like. The polymer peptide can be purified and obtained by conventional methods such as purification from microbial culture broths, enzymatic methods, or chemical synthesis, or they can be purified and used from natural sources such as pig or cattle skin, or from natural materials like silkworm fibroin. As for polymer polysaccharide, any polymer that can be blended into cosmetics is acceptable, but preferably, hydroxyethyl cellulose, xanthan gum, sodium hyaluronate, chondroitin sulfate or its salts (such as sodium salt), etc. can be mentioned. For example, chondroitin sulfate or its salts can be purified and used conventionally from mammals or fish.

As for sphingolipid, any sphingolipid that can be blended into cosmetics is acceptable, but preferably, ceramides, phytosphingosine, sphingoglycolipids, etc. can be mentioned. Sphingolipids can be purified and obtained conventionally from mammals, fish, mollusks, yeast, or plants by conventional purification methods or chemical synthesis.

As for seaweed extracts, any extract that can be blended into cosmetics is acceptable, but preferably, extracts such as kelp extract, red algae extract, brown algae extract, etc. can be mentioned, and from these seaweed extracts, purified carrageenan, alginate, sodium alginate, potassium alginate, etc. used in the present invention can be included. Seaweed extracts can be conventionally purified from seaweed using common methods.

The cosmetics of the present invention may be blended with the above essential ingredients together with other ingredients that are blended in cosmetics normally as necessary. The blending ingredients that may be added may include fat ingredients, moisturizers, emollients, surfactants, organic and inorganic pigments, organic powders, ultraviolet absorbers, preservatives, germicides, antioxidants, plant extracts, pH adjusters, alcohols, pigments, blood flow promoters, cooling agents, restrictors, and purified water. The fat ingredients include ester-based fats, hydrocarbon-based fats, silicon-based fats, fluorine-based fats, animal fats, and plant fats.

Examples of ester-based fats can be tri2-ethylhexanoic acid glyceryl, 2-ethylhexanoic acid cetyl, myristic acid isopropyl, myristic acid butyl, palmitic acid isopropyl, ethyl stearate, palmitic acid octyl, isostearic acid isocetyl, butyl stearate, linoleic acid ethyl, linoleic acid isopropyl, oleic acid ethyl, myristic acid isocetyl, myristic acid isostearyl, palmitic acid isostearyl, myristic acid octyldodecyl, isostearic acid isocetyl, sebacic acid diethyl, adipic acid diisopropyl, neopentanoic acid isoalkyl, tri (capryl, capric acid) glyceryl, tri2-ethylhexanoic acid trimethylolpropane, triisostearic acid trimethylolpropane, tetra2-ethylhexanoic acid pentaerythritol, caprylic acid cetyl, lauric acid decyl, lauric acid hexyl, myristic acid decyl, myristic acid myristyl, myristic acid cetyl, stearyl stearate, oleic acid decyl, ricinoleic acid cetyl, lauric acid isostearyl, myristic acid isotridecyl, palmitic acid isocetyl, octyl stearate, isostearic acid isocetyl, oleic acid isodecyl, oleic acid octyldodecyl, linoleic acid octyldodecyl, isostearic acid isopropyl, 2-ethylhexanoic acid cetostearyl, 2-ethylhexanoic acid stearyl, isostearic acid hexyl, dioctanic acid ethylene glycol, dioleic acid ethylene glycol, dicapric acid propylene glycol, di (capryl, capric acid) propylene glycol, dicapric acid propylene glycol, dicapric acid neopentyl glycol, dioctanic acid neopentyl glycol, tricapric acid glyceryl, triundecyl acid glyceryl, triisopalmitic acid glyceryl, triisostearic acid glyceryl, neopentanoic acid octyldodecyl, octanic acid isostearyl, isononanoic acid octyl, neodecanoic acid hexyldecyl, neodecanoic acid octyldodecyl, isostearic acid isocetyl, isostearic acid isostearyl, isostearic acid octyldecyl, polyglycerine oleic acid ester, polyglycerine isostearic acid ester, triisocetyl citrate, triisoalkyl citrate, triisooctyl citrate, lauric acid, myristyl lactate, cetyl lactate, octyldecyl lactate, triethyl citrate, acetyltriethyl citrate, acetyltributyl citrate, trioctyl citrate, diisostearyl malate, 2-ethylhexyl hydroxystearate, di2-ethylhexyl succinate, diisobutyl adipate, sebacic acid diisopropyl, dioctyl sebacate, cholesteryl stearate, cholesteryl isostearate, cholesteryl hydroxystearate, cholesteryl oleate, dihydrocholesteryl olein acid, pitostearyl isostearate, pitostearyl oleate, 12-steroyl hydroxystearic acid isocetyl, 12-steroyl hydroxystearic acid stearyl, 12-steroyl hydroxystearic acid isostearyl and the like.

Examples of hydrocarbon-based fats can be squalene, liquid paraffin, alpha-olefin oligomer, isoparaffin, ceresin, paraffin, liquid isoparaffin, polybutene, microcrystalline wax, wacellin and the like.

Examples of silicone-based fats can be polymethylsilicone, methylphenylsilicone, methylcyclopolysiloxane, octamethylpolysiloxane, decamethylpolysiloxane, dodecamethylcyclosiloxane, dimethylsiloxane and methylcetyloxysiloxane copolymer, dimethylsiloxane and methylstearoxysiloxane copolymer, alkyl-modified silicone oil, amino-modified silicone oil and the like.

Examples of fluorine-based fats can be perfluoropolyethers and the like.

Examples of animal or plant fats can be animal or plant fats such as avocado oil, armond oil, olive oil, sesame oil, rice bran oil, safflower oil, soybean oil, corn oil, rapeseed oil, apricot kernel oil, palm kernel oil, palm oil, castor oil, sunflower oil, grapeseed oil, cottonseed oil, palm oil, cook ear oil, malt germ oil, rice germ oil, cya butter, walnut oil, marker data nut oil, medo home oil, egg yolk oil, tallow oil, mink oil, mink oil, orange rape oil, jojoba oil, candelir wax, carnauba wax, liquid lanolin, and hardened castor oil.

Examples of moisturizers can be water-soluble low molecular moisturizers, oil-soluble molecular moisturizers, water-soluble polymers and oil-soluble polymers and the like.

Examples of water-soluble low molecular moisturizers can be serine, glutamine, sorbitol, mannitol, pyrrolidone-sodium carboxylate, glycerin, propylene glycol, 1,3-butylene glycol, ethylene glycol, polyethylene glycol B (degree of polymerization n=2 or more), polypropylene glycol (degree of polymerization n=2 or more), polyglycerin B (degree of polymerization n=2 or more), lactic acid and lactic acid salts.

Examples of fat-soluble low molecular moisturizers can be cholesterol and cholesterol esters.

Examples of water-soluble polymers can be carboxyvinyl polymers, polyaspartates, tragacanth, xanthan gum, methylcellulose, hydroxymethylcellulose, hydroxyethylcellulose, hydroxypropylcellulose, carboxymethylcellulose, water-soluble chitin, chitosan and dextrin.

Examples of oil-soluble polymers can be polyvinylpyrrolidone and eicosene copolymers, polyvinylpyrrolidone and hexadecene copolymers, nitrocellulose, dextrin fatty acid ester and polymeric silicones. Examples of emollient include long-chain acyl glutamic acid cholesteryl esters, hydroxystearic acid cholesteryls, 12-hydroxystearic acid, stearic acid, rosin acid and lanolin fatty acid cholesteryl esters.

Examples of surfactants can be nonionic surfactants, anionic surfactants, cationic surfactants and amphoteric surfactants.

Examples of non-ionic surfactants self-emulsifying monostearate glycerin, propylene glycol fatty acid esters, glycerin fatty acid esters, polyglycerin fatty acid esters, sorbitan fatty acid esters, POE (polyoxyethylene) sorbitan fatty acid esters, POE sorbitol fatty acid esters, POE glycerin fatty acid esters, POE alkyl ethers, POE fatty acid esters, POE hardening pimaja oil, POE pimaja oil, POE and POP (polyoxyethylene and polyoxypropylene) copolymers, POE and POP alkyl ethers, polyether-modified silicones, lauric acid alkylolamide, alkyl amine oxides, hydrogenated soybean phospholipids, etc. Examples of anionic surfactants include fatty acid soaps, alpha-alkyl sulfonates, alkyl sulfates, alkyl aryl sulfonates, alkyl naphthalene sulfonates, alkyl sulfonates, POE alkyl ether sulfates, alkyl amide sulfates, alkyl phosphates, POE alkyl phosphates, alkyl amide phosphates, alkylol alkyl taurines, N-acyl amino acids, POE alkyl ether carboxylates, alkyl sulfosuccinates, alkyl sulfacetates, acylated collagen peptide, and perfluoroalkyl phosphate esters.

Examples of cationic surfactants include alkyltrimethylammonium chloride, stearyldimethylammonium chloride, cetyltrimethylammonium bromide, cetylstearyldimethylammonium chloride, distearyldimethylammonium chloride, stearyldimethylbenzylammonium chloride, behenyltrimethylammonium bromide, benzalkonium chloride, stearyl dimethyl ethyl ammonium ethosulfate, stearyl dimethyl amino propylamide, lanolin derivatives quaternary ammonium salts, etc. As positive surfactants, examples include carboxy betaine type, amido betaine type, sulfobetaine type, hydroxy sulfobetaine type, amido sulfobetaine type, phosphobetaine type, amino carboxylate type, imidazoline derivatives, amido amine type, etc.

Examples of organic and inorganic pigments include silica, anhydrous silica, magnesium silicate, talc, sericite, mica, kaolin, bengal, clay, bentonite, titanium dioxide, bismuth oxychloride, zirconium oxide, magnesium oxide, zinc oxide, titanium oxide, aluminum oxide, calcium sulfate, barium sulfate, magnesium sulfate, calcium carbonate, magnesium carbonate, iron oxide, ultramarine, chromium oxide, chromic hydroxide, kaolin, and their complexes as inorganic pigments; polyamide, polyester, polypropylene, polystyrene, polyurethane, vinyl resin, urea resin, phenolic resin, fluororesin, silicone resin, acrylic resin, melamine resin, epoxy resin, polycarbonate resin, divinylbenzene and styrene copolymers, silk powder, cellulose, CI pigment yellow, CI pigment orange, and their complexes with inorganic pigments and organic pigments, etc.

Examples of organic particles include metal soaps such as calcium stearate; alkyl phosphorus metal salts such as zinc cetyl phosphate, zinc lauryl phosphate, and calcium lauryl phosphate; acyl amino acid chelates such as N-lauroyl-beta-alanine calcium, N-lauroyl-beta-alanine zinc, and N-lauroyl glycine calcium; amidosulfonic acid chelates such as N-lauroyl taurate calcium and N-palmitoyl taurate calcium; N-acyl amino acids such as N-epsilon-lauroyl-L-lysine, N-epsilon-palmitoyllysine, N-alpha-palmitoylolethanolamine, N-alpha-lauroylarginine, and N-alpha-hardening saturated fatty acylarginine; N-acylated amino acid peptides such as N-lauroyl glycylglycine; alpha-amino fatty acids such as alpha-amino caprylic acid and alpha-amino lauric acid; and materials such as polyethylene, polypropylene, nylon, polymethyl methacrylate, polystyrene, divinylbenzene and styrene copolymers, and polytetrafluoroethylene.

Examples of ultraviolet absorbers include paraaminobenzoic acid, paraaminobenzoic acid ethyl, paraaminobenzoic acid amyl, paraaminobenzoic acid octyl, salicylic acid ethylene glycol, saliinic acid phenyl, saliinic acid octyl, saliinic acid benzyl, saliinic acid butylphenyl, saliinic acid homomentyl, cinnamic acid benzyl, paramethoxycinnamic acid-2-ethoxyethyl, paramethoxycinnamic acid octyl, diparamethoxycinnamic acid octyl-2-ethylhexaneglyceryl, paramethoxycinnamic acid isopropyl, diisopropyl and diisopropyl cinnamic acid ester mixtures, urocanic acid, urocanic acid ethyl, hydroxymethoxybenzophenone, hydroxymethoxybenzophenonesulfonic acid and its salts, dihydroxymethoxybenzophenone, dihydroxymethoxybenzophenone disulfonic acid sodium, dihydroxybenzophenone, tetrahydroxybenzophenone, 4-tert-butyl-4′-methoxydibenzoylmethane, 2,4,6-trianilino-p-(carbo-2′-ethylhexyl-1′-oxy)-1,3,5-triazine, and 2-(2-hydroxy-5-methylphenyl) benzotriazole.

Disinfectants include hinokitiol, triclosan, trichlorohydroxydiphenyl ether, chlorhexidine gluconate, phenoxyethanol, resorcin, isopropylmethylphenol, azulene, salicylic acid, zincphyllithione, benzalkonium chloride, Examples include photosensitizer No. 301, sodium mononitroguaiacol, and undecirenic acid.

Examples of antioxidants include butylhydroxyanisole, propyl gallate, and elisorbic acid.

Examples of pH adjusting agents include citric acid, sodium citrate, malic acid, sodium maleate, fumaric acid, sodium fumarate, succinic acid, sodium succinate, sodium hydroxide, sodium monohydrogen phosphate.

Alcohols include higher alcohols such as cetyl alcohol.

In addition, the blending ingredients that may be added otherwise are not limited thereto, and any of the above ingredients may be blended within the scope that does not impair the purpose and effect of the present invention.

The cosmetics of the present invention may take the formulation of a solution, an emulsion, a viscous mixture, etc.

The ingredients included in the cosmetic composition of the present invention may include ingredients commonly used in the cosmetic composition as active ingredients, for example, common adjuvants and carriers such as stabilizers, solubilizers, vitamins, pigments, and fragrances.

The cosmetic composition for improving muscle function of the present invention may be prepared in any formulation commonly prepared in the art, for example, emulsions, creams, cosmetic water, packs, foundations, lotions, cosmetic solutions, hair cosmetics, etc.

Specifically, the cosmetic composition of the present invention includes formulations of skin lotions, skin softers, skin toners, milk lotions, astringents, lotions, moisturizer lotions, nutrition lotions, taste creams, nutrition creams, moisturizer creams, hand creams, foundations, essences, nutrition essences, packs, soaps, cleansing foams, cleansing lotions, cleansing creams, hair lotions, hairtonics, hair essences, hair shampoos, hair rinses, hair treatments, body lotions, and body cleansers.

In the case of the present invention being in the form of paste, cream, or gel, the base components may include animal fibers, plant fibers, waxes, paraffin, starch, tragacanth, cellulose derivatives, polyethylene glycol, silicone, bentonite, silica, talc, or zinc oxide.

In the case of the present invention being in the form of powder or spray, as the carrier ingredient, lactose, talc, silica, aluminum hydroxide, calcium silicate, or polyamide powder may be used, and particularly, especially in the case of sprays, additional propellants such as chlorofluorohydrocarbons, propane/butane, or dimethyl ether may be included.

In the case of the present invention being in the form of solution or emulsion, as the carrier ingredient, solvents, solvating agents, or emulsifying agents may be used, and examples thereof include water, ethanol, isopropanol, ethyl carbonate, ethyl acetate, benzyl alcohol, benzyl benzoate, propylene glycol, 1,3-butyl glycol oil, glycerol aliphatic ester, polyethylene glycol, or fatty acid esters of sorbitan.

In the case of the present invention being in the form of a suspension, as the carrier ingredient, liquid diluents such as water, ethanol or propylene glycol, suspending agents such as ethoxylated isostearyl alcohol, polyoxyethylene sorbitol ester, and polyoxyethylene sorbitan ester, microcrystalline cellulose, aluminum metahydroxide, bentonite, agar, or tragacanth, etc.

In the case of the present invention being in the form of a surfactant-containing cleanstaining, as the carrier ingredient, aliphatic alcohol sulfate, aliphatic alcohol ether sulfate, sulfosuccinic acid monoester, isethionate, imidazolinium derivative, methyltaurate, sarcosinate, fatty acid amide ether sulfate, alkylamidobetaine, aliphatic alcohol, fatty glyceride, fatty diethanolamide, vegetable oil, linoline derivative, or ethoxylated glycerol fatty acid ester, etc.

In addition, the present invention provides a method for treating muscle disorders, comprising administering a pharmacologically effective amount of Sargassum serratifolium extract to a subject.

The therapeutic method of the present invention includes administering the active ingredient in a therapeutically effective amount to a subject. It is preferable that the specific therapeutically effective amount for a specific subject be applied differently depending on various factors including the type and degree of response to be achieved, the specific composition including whether other agents are used, the age, body weight, general health status, sex and diet of the subject, the time of administration, the route of administration and the secretion rate of the composition, the treatment period, the drugs used together with or used simultaneously with the specific composition, and similar factors well known in the pharmaceutical art. The daily dose is 0.0001 to 100 mg/kg, preferably 0.01 to 100 mg/kg, based on the amount of the pharmaceutical composition of the present invention, and may be administered 1 to 6 times per day. However, it is apparent to those skilled in the art that the dosage or administration of each active ingredient should be such that side effects are not caused by including an excessively high content of each active ingredient. Therefore, it is preferable that the effective amount of the composition suitable for the purpose of the present invention is determined in consideration of the aforementioned matters.

The subject may be applied to any mammal, and the mammal includes humans and primates, as well as domestic animals such as cattle, pigs, sheep, horses, dogs and cats.

The active ingredient of the present invention can be administered to mammals such as rats, mice, livestock, and humans through various routes. All modes of administration are contemplated, for example, orally, rectally or intravenously, intramuscularly, subcutaneously, intrathecally or by injection.

### MODES OF THE INVENTION

Hereinafter, the present invention will be described in more detail with reference to the following Examples. However, the following Examples are only intended to embody the contents of the present invention, and the present invention is not limited thereto.

### Example 1. Preparing the Sargassum serratifolium Ethanol Extract

Sargassum serratifolium, naturally growing in Gijang-gun, Busan, and Tongyeong City, Gyeongsangnam-do, was collected using scuba diving. Species identification for specimens collected in Gijang was conducted by Professor Choi Chang-geun of the Department of Ecological Engineering at Pukyong National University, and species identification for those collected in Tongyeong was performed by Professor Kim Nam-gil of the Department of Marine Bioscience at Gyeongsang National University. After naturally drying the Sargassum serratifolium, 1.5 kg of the powder obtained by grinding was placed into 6 L of ethanol (85%) and reflux-extracted at approximately 70° C. for about 3 hours, repeated twice. After refluxing and cooling, the extract was filtered to remove impurities using a tangential flow filtration system (MWCO 50 kDa). The filtrate was concentrated using a vacuum rotary evaporator at 40° C. to remove the ethanol, yielding 160 g of extracted residue as the subextract. The obtained Sargassum serratifolium ethanolic extract was stored refrigerated at 4° C. for subsequent use as a sample.

### Example 2. Confirmation of Cytotoxicity of the Sargassum serratifolium Ethanol Extract

In order to confirm the concentration of the Sargassum serratifolium ethanol extract that is not toxic to C2C12 myotubes, MTS analysis was performed.

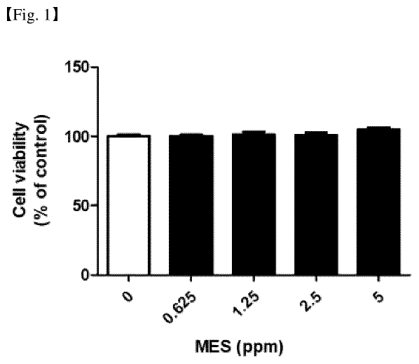

The results showed that the viability of C2C12 cells was not affected up to a concentration of 5 ppm of the Sargassum serratifolium ethanol extract (FIG. 1).

### Example 3. The Effect of Inhibiting Muscle Atrophy of the Sargassum serratifolium Ethanol Extract

### 3-1. Cell Protection Effect on Muscle Atrophy by Dexamethasone

To investigate the effects of the Sargassum serratifolium ethanol extract on C2C12 myotubes with dexamethasone-induced atrophy, an MTS assay was conducted.

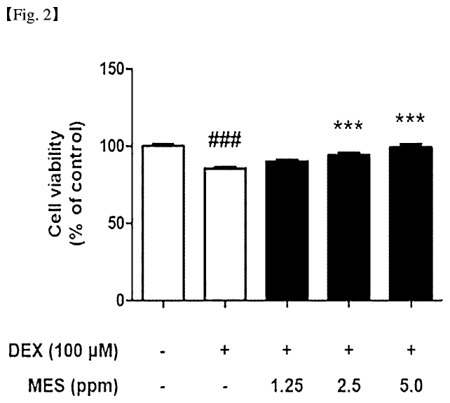

The results showed that the Sargassum serratifolium ethanol extract inhibited the decrease in cell viability caused by dexamethasone (FIG. 2). This indicates that the Sargassum serratifolium ethanol extract has a protective effect on C2C12 cells against dexamethasone.

### 3-2. Effect of Improving in Myotube Diameter Reduction

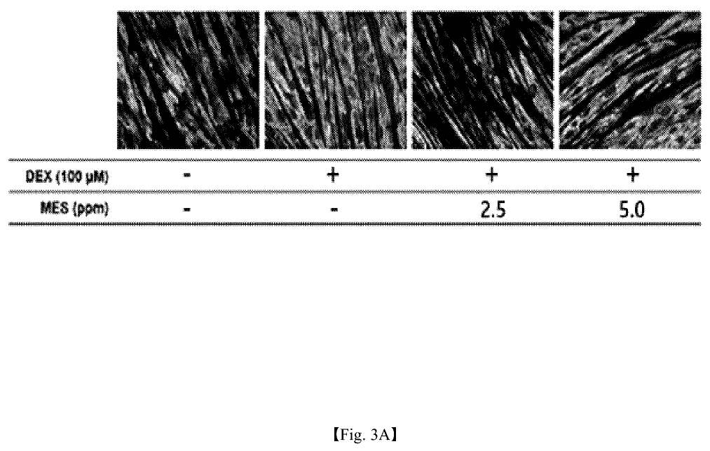

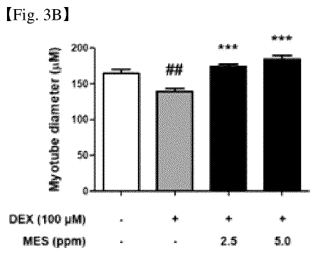

In order to confirm whether the Sargassum serratifolium ethanolic extract inhibits muscle atrophy in dexamethasone-induced C2C12 myotubes, the extent of inhibition was evaluated based on the reduction in myotube diameter using Giemsa-Wright staining. The results revealed that the Sargassum serratifolium ethanolic extract alleviated the decrease in myotube diameter induced by dexamethasone (FIGS. 3A and 3B).

### Example 4. Verification of the Mechanism of Muscle Atrophy Inhibition of the Sargassum serratifolium Ethanol Extract

### 4-1. Identification of Muscle Hypertrophy Pathway

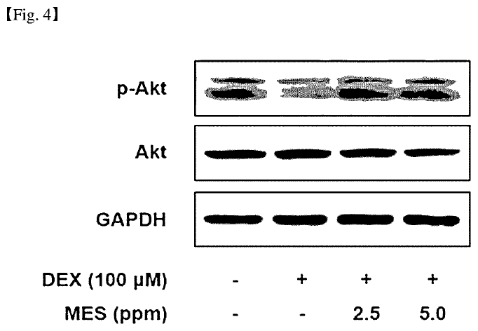

In order to confirm the effect of the Sargassum serratifolium ethanol extract on the regulation of Akt activation, which induces muscle hypertrophy/atrophy, in C2C12 myotubes with dexamethasone-induced atrophy, the phosphorylation levels of Akt was examined using Western blot analysis. The results showed that the phosphorylation levels of Akt, which were reduced by dexamethasone, were increased by treatment with the Sargassum serratifolium ethanol extract (FIG. 4). This indicates that the Sargassum serratifolium ethanol extract inhibits muscle atrophy and induces muscle hypertrophy through the muscle hypertrophy pathway.

### 4-2. Confirmation of the Lower Pathway

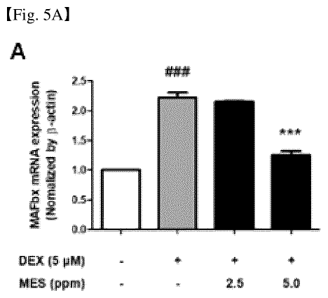

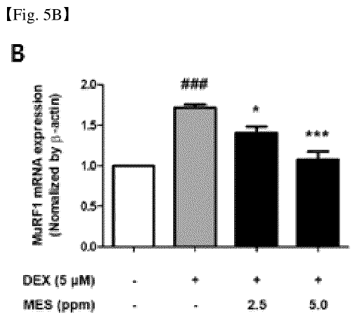

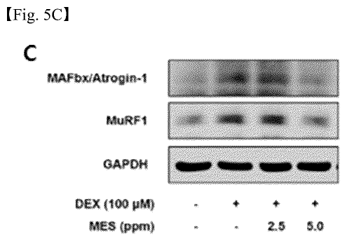

When the activation of Akt is reduced, the expression of muscle-specific E3 ubiquitin ligases, including atrogin-1/MAFbx and MuRF1, is promoted via FoxO, leading to muscle protein degradation. Therefore, in dexamethasone-induced C2C12 myotubes, the mRNA and protein expression levels of atrogin-1/MAFbx and MuRF1 were examined using qPCR and Western blot analysis. The results showed that the mRNA (FIGS. 5A and 5B) and protein expression levels (FIG. 5C) of atrogin-1/MAFbx and MuRF1 were significantly reduced by the Sargassum serratifolium ethanol extract. This confirms the inhibitory effect of the Sargassum serratifolium ethanol extract on muscle protein degradation in muscle atrophy.

### Example 5. Inhibitory Effect of Sargassum serratifolium Ethanol Extract on Atorvastatin-Induced Muscle Atrophy

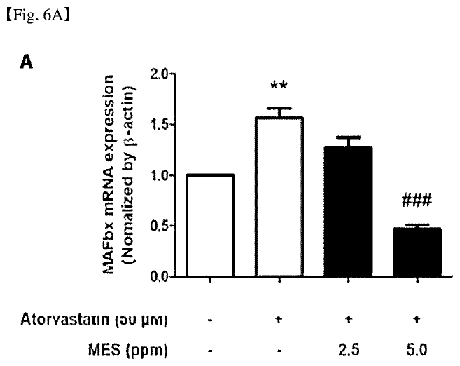

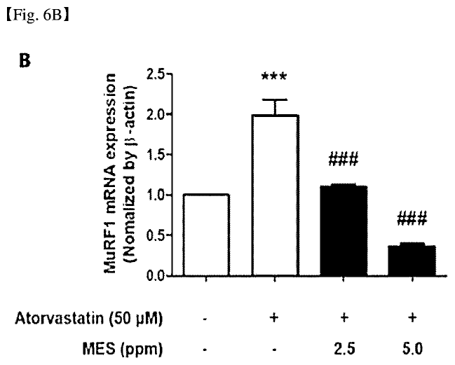

Due to reported muscle loss as a side effect of the lipid-lowering drug Atorvastatin, the effect of Sargassum serratifolium ethanol extract on Atorvastatin-induced muscle atrophy in C2C12 myotubes was examined by qPCR to conform the changes in the mRNA expression levels of atrogin-1/MAFbx and MuRF1, which are related to muscle protein degradation. The results showed that the mRNA expression levels of atrogin-1/MAFbx and MuRF1 are significantly decreased by treating the Sargassum serratifolium ethanol extract (FIGS. 6A and 6B).

### Example 6. Preparation of Muscle Atrophy Animal Model and Confirmation of Body Weight Change

To confirm the improvement effect of the Sargassum serratifolium ethanol extract on muscle atrophy, a muscle atrophy mouse model was prepared. Specifically, three-week-old male C57BL/6 mice were provided with free access to water and food and were acclimated under conditions of 23±2° C., relative humidity of 50±5%, and a 12-hour light/dark cycle. After 8 weeks of acclimation, the mice were randomly divided into the following groups: untreated group (CON, n=7), dexamethasone-treated group (DEX, n=7), dexamethasone and Sargassum serratifolium ethanol extract 30 mg/kg/day group (MES30, n=7), dexamethasone and Sargassum serratifolium ethanol extract 60 mg/kg/day group (MES60, n=8), and dexamethasone and Sargassum serratifolium ethanol extract 120 mg/kg/day group (MES120, n=8). The Sargassum serratifolium ethanol extract was dissolved in olive oil, and the control and dexamethasone-treated groups received the same volume of olive oil. The Sargassum serratifolium ethanol extract was administered orally every day for 1 week before the dexamethasone injection and continued to be administered orally during the 5-week experimental period. To induce muscle atrophy, dexamethasone (10 mg/kg/day) was administered intraperitoneally once daily for 4 weeks, starting one week after the administration of Sargassum serratifolium ethanol extract. In the untreated group, an equivalent volume of saline was administered intraperitoneally. Body weight and food intake were evaluated daily. The experiment was conducted with the approval of the Pukyong National University Animal Care and Use Committee, in accordance with Pukyong National University's animal experiment ethics regulations. In the muscle atrophy-induced mice, body weight gain was calculated using the body weight on the first day of dexamethasone administration and the body weight at the time of sacrifice, and the results are shown in Table 1. below.

TABLE 1

| | Body weight (g) | | |
|---|---|---|---|
| | At the first | | Body weight gain during the test |
| Group | administration [A] | Sacrifice [B] | article treatment period (g) [B − A] |
| CON | 31.4 ± 1.98 | 29.6 ± 2.29 | −1.8 ± 2.29 |
| DEX | 30.4 ± 3.18 | 27.9 ± 3.25 | −6 ± 3.25 |
| DEX + MES30 | 30 ± 2.25 | 28.4 ± 2.82 | −1.9 ± 2.82 |
| DEX + MES60 | 29.7 ± 3.11 | 29.1 ± 2.94 | −0.6 ± 2.94 |
| DEX + MES120 | 29.2 ± 4.19 | 29.4 ± 4.8 | 0.2 ± 4.8 |

As a result, as shown in Table 1 above, the DEX group treated with dexamethasone showed a significant decrease in body weight at the sacrifice time compared to the CON group. However, it was observed that the weight loss was inhibited in the group treated with Sargassum serratifolium extract.

### Example 7 Confirmation of the Effect of Improving Muscle Atrophy of Sargassum serratifolium Ethanol Extract (In Vivo)

### 7-1. Confirmation of the Effect of Increasing Muscle Strength

To confirm the effect of improving muscle atrophy of the Sargassum serratifolium ethanol extract, Grip strength test and Weights test were used to assess the effect of improving muscle strength. Specifically, the grip strength test was conducted in the 5th week of the drug administration to measure the forelimb strength using a grip strength meter. The forelimbs of each mouse were placed on the grip strength meter's grid bar, and their tails were gently pulled back to keep their bodies horizontal. This process was repeated 10 times at 10-second intervals to measure the average grip strength.

Additionally, the weights test was conducted in the 5th week of the drug administration to measure forelimb muscle strength using weights. Starting with the lightest weight, 23 g, 30 g, 38 g, 44 g, 50 g, 58 g, 68 g, 78 g, 94 g, and 110 g weights were used. Each mouse attempted to lift each weight with its forelimbs and hold it for more than 3 seconds before moving on to the next heavier weight. The maximum weight each mouse could lift was recorded.

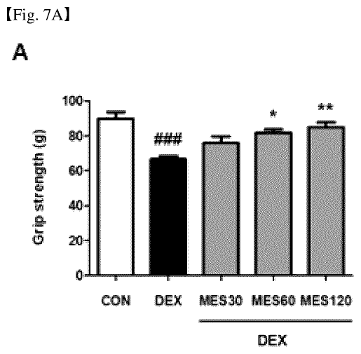

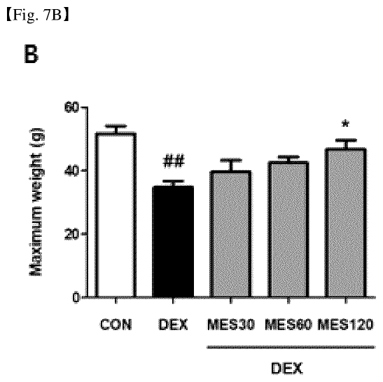

As a result, as shown in FIG. 7, in the grip strength test, the DEX group exhibited a significant decrease in grip strength compared to the CON group. However, in the MES groups administered with Sargassum serratifolium ethanol extract, grip strength increased in a concentration-dependent manner (FIG. 7A). Similarly, in the weights test, the average weight lifted by the DEX group was significantly reduced compared to the CON group, indicating decreased muscle strength. Conversely, in the MES groups, the average weight lifted increased significantly in a concentration-dependent manner (FIG. 7B), indicating improved muscle strength.

7-2. Confirmation of the Effect of Increasing Muscle Function of Sargassum serratifolium Ethanol Extract

In order to confirm whether the Sargassum serratifolium extract of the present invention increases muscle performance (muscle endurance), a treadmill test was conducted. Specifically, mice from each group in Example 6 were evaluated using running on a treadmill. The evaluation was performed on a treadmill inclined at an angle (∠R=10°), starting at 10 m/min for 20 minutes and gradually increasing by 2 m/min every 2 minutes. If a mouse stayed on the shock grid for 10 seconds, it was considered fatigued, and the experiment was terminated. The running distance (m), running time (min), and maximum speed (m/min) were measured.

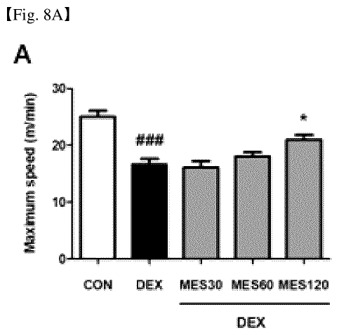

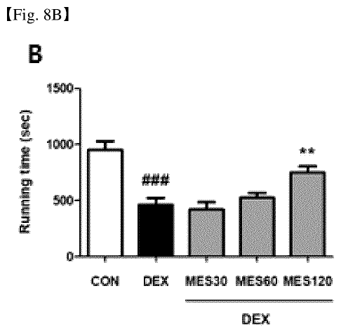

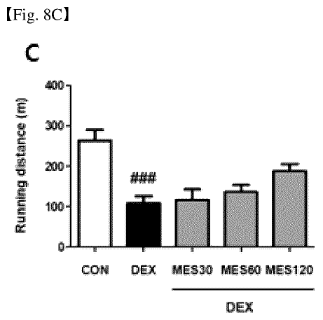

As a result, as shown in FIG. 8, compared to the CON group, the DEX group showed significant decreases in running distance, running time, and maximum speed. However, in the MES group treated with the Sargassum serratifolium ethanol extract, it was confirmed that the maximum speed (FIG. 8A), running time (FIG. 8B), and running distance (FIG. 8C) increased in a concentration-dependent manner, indicating that the Sargassum serratifolium ethanol extract improves muscle function.

### 7-3. Measurement of Fat Mass, Lean Mass, and Calf Thickness.

The fat and lean body mass of the mice in each group were measured using Dual Energy X-ray Absorptiometry (DEXA) just before sacrifice. Specifically, in the muscle atrophy mouse model, the ratios of lean body mass to total mass and fat mass to total mass were checked to observe changes in lean body mass and fat mass. Additionally, the thickness of the calf was measured using an electronic digital caliper on the left hind leg after exposing the muscles post-sacrifice.

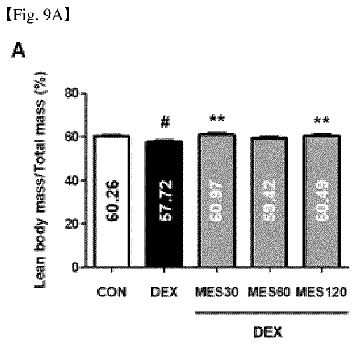

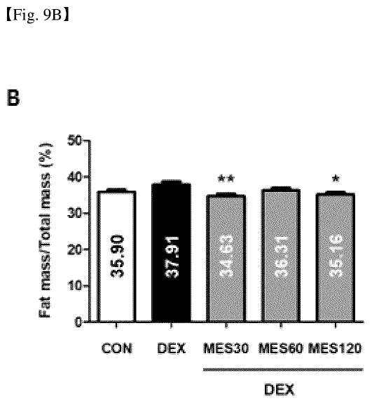

As shown in FIG. 9A, compared to the CON group, the DEX group showed a decrease in the ratio of lean body mass/total mass, indicating a reduction in weight excluding fat and induction of muscle atrophy. However, in the MES group treated with the Sargassum serratifolium ethanol extract, the reduced ratio of lean body mass/total mass increased, indicating an increase in weight excluding fat and an increase in muscle mass.

In addition, compared to the CON group, the DEX group showed an increase in the ratio of fat mass/total mass, indicating an increased proportion of fat within the body weight. However, in the MES group treated with the Sargassum serratifolium ethanol extract, the increased ratio of fat mass/total mass was significantly decreased, confirming a reduction in the proportion of fat within the body weight (FIG. 9B).

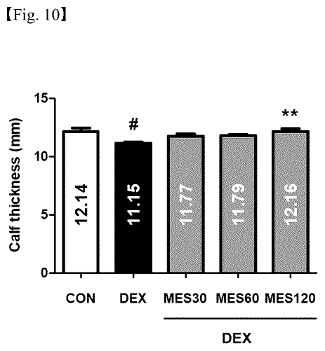

Furthermore, to assess calf muscle mass, calf thickness was measured. Compared to the CON group, the DEX group showed a significant decrease in calf thickness. However, in the MES group treated with the Sargassum serratifolium ethanol extract, the decreased calf thickness was significantly increased, confirming that the Sargassum serratifolium ethanol extract improves muscle atrophy and increases muscle mass (FIG. 10).

### 7-4. Statistical Analysis

All data in the present invention were expressed as mean #SEM of at least seven independent experiments, and significance between groups was evaluated using one-way analysis of variance followed by Tukey's multiple comparison test using GraphPad Prism 5.0.

Therefore, the Sargassum serratifolium extract of the present invention has been confirmed to protect muscle cells from dexamethasone-induced muscle atrophy caused by the side effects of atorvastatin, inhibit myotube diameter reduction, inhibit muscle atrophy, induce muscle hypertrophy, and inhibit muscle protein degradation. Additionally, in animal models with dexamethasone-induced muscle atrophy, it has been confirmed to increase muscle strength, improve muscle function, and increase muscle mass, thereby improving muscle diseases.

1. A pharmaceutical composition for preventing or treating muscle disease comprising Sargassum serratifolium extract as an active ingredient.

2. The composition of claim 1, wherein the Sargassum serratifolium extract is extracted with at least one solvent selected from the group consisting of water, an organic solvent having 1 to 6 carbon atoms, a subcritical fluid, and a supercritical fluid.

3. The composition of claim 2, wherein the organic solvent having 1 to 6 carbon atoms is at least one solvent selected from the group consisting of alcohol, acetone, ether, benzene, chloroform, ethyl acetate, methylene chloride, hexane, cyclohexane, and petroleum ether.

4. The composition of claim 1, wherein the muscle disease is at least one selected from the group consisting of atony, muscular atrophy, muscular dystrophy, muscle degeneration, muscle asthma, cachexia, and sarcopenia.

5. The composition of claim 1, wherein the Sargassum serratifolium extract protects muscle cells.

6. The composition of claim 1, wherein the Sargassum serratifolium extract inhibits the myotube diameter reduction.

7. The composition of claim 1, wherein the Sargassum serratifolium extract increases phosphorylation of Akt.

8. The composition of claim 1 wherein the Sargassum serratifolium extract inhibits mRNA and protein expression of atrogin-1/MAFbx or MuRF1.

9. The composition of claim 1, wherein the Sargassum serratifolium extract increases muscle strength.

10. The composition of claim 9, wherein increasing the muscle strength refers to increasing the grip strength.

11. The composition of claim 1, wherein the Sargassum serratifolium extract improves muscle function.

12. The composition of claim 11, wherein the muscle function is muscle endurance.

13. The composition of claim 1, wherein the Sargassum serratifolium extract increases muscle mass compared to body weight.

14. A food composition for preventing or improving muscle disease comprising Sargassum serratifolium extract as an active ingredient.

15. The composition of claim 14, wherein the Sargassum serratifolium extract is extracted with at least one solvent selected from the group consisting of water, an organic solvent having 1 to 6 carbon atoms, a subcritical fluid, and a supercritical fluid.

16. The composition of claim 15, wherein the organic solvent having 1 to 6 carbon atoms is at least one solvent selected from the group consisting of alcohol, acetone, ether, benzene, chloroform, ethyl acetate, methylene chloride, hexane, cyclohexane, and petroleum ether.

17. The composition of claim 14, wherein the muscle disease is at least one selected from the group consisting of atony, muscular atrophy, muscular dystrophy, muscle degeneration, muscle asthma, cachexia, and sarcopenia.

18-23. (canceled)

24. A method for treating muscle diseases comprising:
administering a pharmaceutically effective amount of Sargassum serratifolium extract to a subject in need thereof.
